(19) United States
(12) Patent Application Publication
(10) Pub. No.: US 2024/0382597 A1
(43) Pub. Date: Nov. 21, 2024

(54) FORMULATIONS OF ANTI-INFLAMMATORY AGENTS COMPRISING MSM WITH ENHANCED SOLUBILITY IN WATER

(71) Applicant: LIVIONEX, Inc., Los Gatos, CA (US)

(72) Inventors: Rajiv BHUSHAN, Los Gatos, CA (US); Jerry GIN, Sunnyvale, CA (US); Amit GOSWAMY, Los Gatos, CA (US)

(21) Appl. No.: 18/270,724

(22) PCT Filed: Dec. 31, 2021
(86) PCT No.: PCT/US2021/065850

§ 371 Date: Jun. 30, 2023

(51) Int. Cl. A61K 47/20; A61K 9/06; A61K 31/192; A61K 31/196; A61K 31/573; A61K 31/618

(52) U.S. Cl. CPC A61K 47/20; A61K 31/192; A61K 31/196; A61K 31/573; A61K 31/618; A61K 9/06

(57) ABSTRACT

Pharmaceutical formulations comprising anti-inflammatory and/or pain-reducing agents that are insoluble or have low solubility in water are formulated with a solubility enhancer such as MSM to increase solubility. The enhanced solubility of the agents in the presence of MSM allows use of reduced amounts of drugs and thus significantly increases efficiency and reduces harmful side-effects associated with these agents.

### CROSS-REFERENCE TO RELATED APPLICATIONS

This application is a 371 National Stage filing of an international application filed under the Paris Convention Treaty (PCT) No. PCT/US2021/065850 filed Dec. 31, 2021 titled FORMULATIONS OF ANTI-INFLAMMATORY AGENTS COMPRISING MSM WITH ENHANCED SOLUBILITY IN WATER, the contents of which are incorporated herein in their entirety.

### TECHNICAL FIELD OF THE INVENTION

This disclosure relates generally to the field of pharmacotherapy, and more particularly relates to methods and formulations for use in the treatment of inflammation, pain and wounds. Specifically, the invention relates to anti-inflammatory, pain relieving and wound healing agents with poor solubility in water that are formulated with a solubility-enhancing agent such as methyl sulfonyl methane (MSM) for enhancing solubility of the drugs in water.

### BACKGROUND OF THE INVENTION

Inflammation is initiated as a protective response by the host, but it also can result in systemic pathologies. Inflammation is a complex biological response of vascular tissue to harmful stimuli, such as oxidative stress, irritants, pathogens, and damaged cells. It is a protective attempt by the organism to remove an injurious stimulus and initiate the healing process for injured tissue. The inflammatory response involves the production and release of inflammatory modulators that function to both destroy damaged cells and heal injured tissue. In order to perform this function, however, various inflammatory modulators either directly produce and/or signal the release of agents that produce reactive oxygen species for the purpose of destroying invading agents and/or injured cells.

Local anesthetics are used in many medical procedures in order to prevent or relieve pain, itching, and burning for a temporary period of time through the blocking of nerve signals and are advantageous when rapid relief is needed. Examples of procedures that local anesthetics are used in include dental procedures such as teeth cleaning and filling cavities, and minor surgeries. Local anesthetics are of two different types, aminoamides and aminoesters. The aminoamides include articaine, bupivacaine, cinchocaine, etidocaine, levobupivacaine, lidocaine, mepivacaine, prilocaine, ropivacaine, and trimecaine. The amino-esters include benzocaine, chloroprocaine, cyclomethycaine, dimethocaine, piperocaine, propoxycaine, procaine, proparacaine, and tetracaine. Local anesthetics can be used topically or injected.

Although acute pain in response to injury is an important mechanism for reducing the extent of harm to an individual, the nervous system can undergo an adaptive change that results in pain that is perceived well beyond the time after the injury is healed (chronic pain) (Costigan et al., 2009). This chronic pain can be generated by a stimulus that is normally innocuous (allodynia), or the response to a noxious stimulus can be greatly exaggerated (hyperalgesia). Chronic pain is estimated to affect at least 100 million adults in the United States and can negatively affect quality of life.

While inflammation is initiated as a protective response by the host, it often can result in systemic pathologies. Chronic wounds represent a significant burden to patients, health care professionals, and the U.S. health care system, affecting 5.7 million patients and costing an estimated 20 billion dollars annually. Inflammatory lung diseases such as asthma, allergy and chronic obstructive pulmonary disease (COPD) are increasing in developed countries, resulting in great consequences to healthcare costs. Chronic inflammation is associated with normal and pathological ageing. Systemic chronic inflammation can accelerate ageing (Jurk D, et al., Nat Comm, 2014; doi: 10.1038/ncomms5172). Many age-related diseases and ageing itself are closely associated with low-level chronic inflammation (Chung H Y, et al., Ageing Res Rev 2009; 8:18-30).

Rheumatoid arthritis (RA) is a chronic inflammatory disease that affects ˜1% of the population. It is a complicated auto-immune disease and its etiology is unclear, however there are a number of genetic, environmental and other factors that contribute to the pathology. Several treatments are already known to treat an inflammatory disease such as RA. In the case of confirmed diagnosis the recommended gold standard is to initiate methotrexate (MTX) treatment. Methotrexate is the most commonly prescribed drug for rheumatoid arthritis. MTX is often used in combination with other conventional disease modifying anti-rheumatic drugs (DMARDs) and/or corticosteroids.

An anti-inflammatory agent is a drug or substance that reduces inflammation (redness, swelling, and pain) in the body. Anti-inflammatory agents block certain substances in the body that cause inflammation. They are used to treat many different conditions. Such agents are used for the treatment of inflammation and disorders, diseases, and adverse conditions, i.e., pathologies, caused by or otherwise associated with inflammatory processes. The use of both over-the-counter and prescription nonsteroidal medications is frequently recommended in a typical medical practice. But persistent long-term use safety concerns must be considered when prescribing these medications for chronic and degenerative pain conditions.

Prostaglandins are a family of chemicals that are produced by the cells of the body and have several important functions. They promote inflammation that is necessary for healing, but also results in pain, and fever. Prostaglandins are produced within the body's cells by the enzyme cyclooxygenase (COX). Nonsteroidal anti-inflammatory drugs (NSAIDs) block the COX enzymes (COX-1 and COX-2) and reduce prostaglandins throughout the body. As a consequence, ongoing inflammation, pain, and fever are reduced. Examples of FDA approved NSAIDs are aspirin, celecoxib (CELEBREX®), diclofenac (CAMBIAR, CATAFLAM®, VOLTAREN-XR®, ZIPSOR®, ZORVOLEX®), etodolac (LODINE®), ibuprofen (MOTRIN®, Advil®), indomethacin (INDOCIN®), ketoprofen, naproxen (ALEVER, ANAPROX®, NAPRELAN®, NAPROSYN®), oxaprozin (DAYPRO®), piroxicam (FELDENE®), salsalate (DISALSATE®) and tolmetin (TOLECTIN®).

The use of penetration enhancers has been proposed in order to achieve a sufficient and consistent availability of various therapeutic compounds at the site of action during the treatment of cutaneous diseases. For example, U.S. Pat. No. 5,326,566 describes a composition of a pharmacological agent in combination with dibutyl adipate, or a mixture of dibutyl adipate and isopropyl myristate, which could enhance the penetration through the skin and even increase the amount absorbed into the systemic circulation, if that is desired.

A variety of penetration enhancers have been used for enhancing the absorption of therapeutic agents into and through the skin, substantial problems may arise when the penetration enhancers are incompatible with a particular drug substance, leading to drug instability and degradation into potentially harmful degradants.

US 2005/0079210 A1 proposes the use of liposomes the epicutaneous administration of drugs and cosmetically useful agents. However, liposomes are difficult to manufacture cost-effectively and in a reproducible manner.

Unfortunately, most anti-inflammatory and/or pain-reducing agents are insoluble or has low solubility in water, have poor solubility in water, which slows down the transport of these agents in the body, and thereby delays the relief afforded by such agents.

Therefore, there is a need for formulations and methods for increasing the solubility of anti-inflammatory agents for better and quicker treatment and prevention of acute and chronic inflammatory diseases, chronic inflammation, pain, chronic wound healing and burn treatment.

### SUMMARY OF THE INVENTION

The present invention provides formulations that increase solubility of anti-inflammatory agents in water in order to enhance therapy of inflammatory conditions including, but not limited to pain, wound healing and chronic inflammatory diseases.

The present invention further relates to localized uses of a topical treatment to be used in the treatment of chronic wounds. In some embodiments, the topical treatment can be administered to subjects suffering from acute wounds and other soft tissue damage.

In one aspect of the invention, methods and formulations are provided for treatment of pain and treatment of chronic (and acute) wounds in a subject. The method involves administering to the subject an effective amount of a formulation composed of a therapeutically effective amount of an anti-inflammatory agent and an effective amount of a solubility-enhancer having the formula (I)

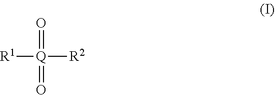

wherein R¹ and R² are independently selected from C₂-C₆ alkyl, C₁-C₆ heteroalkyl, C₆-C₁₄ aralkyl, and C₂-C₁₂ heteroaralkyl, any of which may be substituted, and Q is S or P, wherein the solubility enhancer is present in an amount effective to enhance solubility of the anti-inflammatory agentsuch that the anti-inflammatory agent is delivered in an amount effective to treat chronic wound or burn condition. The solubility enhancing agent may be, for example, methylsulfonylmethane (also referred to as methylsulfone, dimethylsulfone, and DMSO₂), and the anti-inflammatory agent is an NSAIDS and the like.

The invention further provides formulations for use in the aforementioned methods.

These and other aspects will become apparent from the following description of the preferred embodiment taken in conjunction with the following drawings, although variations and modifications therein may be affected without departing from the spirit and scope of the novel concepts of the disclosure.

### DETAILED DESCRIPTION OF THE INVENTION

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The inventions of these publications, patents and published patent applications referenced in this application are hereby incorporated by reference in their entireties into the present invention. Citation herein of a publication, patent, or published patent application is not an admission the publication, patent, or published patent application is prior art.

As used herein and in the appended claims, the singular forms “a,” “and,” and “the” include plural referents unless the context clearly dictates otherwise. Thus, for example, “a solubility enhancer” encompasses a plurality of solubility enhancers as well as a single solubility enhancer. Reference to “a anti-inflammatory agent” includes reference to two or more anti-inflammatory agents as well as a single anti-inflammatory agent, and so forth. In this specification and in the claims that follow, reference will be made to a number of terms, which shall be defined to have the following meanings:

“Optional” or “optionally present”—as in an “optional substituent” or an “optionally present additive” means that the subsequently described component (e.g., substituent or additive) may or may not be present, so that the description includes instances where the component is present and instances where it is not.

By “pharmaceutically acceptable” is meant a material that is not biologically or otherwise undesirable, e.g., the material may be incorporated into a formulation of the invention without causing any undesirable biological effects or interacting in a deleterious manner with any of the other components of the dosage form formulation. However, when the term “pharmaceutically acceptable” is used to refer to a pharmaceutical excipient, it is implied that the excipient has met the required standards of toxicological and manufacturing testing and/or that it is included on the Inactive Ingredient Guide prepared by the U.S. Food and Drug Administration. As explained in further detail infra, “pharmacologically active” (or simply “active”) as in a “pharmacologically active” derivative or analog refers to derivative or analog having the same type of pharmacological activity as the parent agent. The terms “treating” and “treatment” as used herein refer to reduction in severity and/or frequency of symptoms, elimination of symptoms and/or underlying cause, prevention of the occurrence of symptoms and/or their underlying cause, and improvement or remediation of an undesirable condition or damage. Thus, for example, “treating” a subject involves prevention of an adverse condition in a susceptible individual as well as treatment of a clinically symptomatic individual by inhibiting or causing regression of the condition. The term “anti-inflammatory agent” (or “active agent”) refers to any chemical compound, complex or composition that exhibits a desirable effect in the biological context, i.e., when administered to a subject or introduced into cells or tissues in vitro. The term includes pharmaceutically acceptable derivatives of those active agents specifically mentioned herein, including, but not limited to, salts, esters, amides, prodrugs, active metabolites, isomers, analogs, crystalline forms, hydrates, and the like. When the term “anti-inflammatory agent” is used, or when a particular anti-inflammatory agent is specifically identified, it is to be understood that pharmaceutically acceptable salts, esters, amides, prodrugs, active metabolites, isomers, analogs, etc. of the agent are intended as well as the agent per se.

By an “effective” amount or a “therapeutically effective” amount of an active agent is meant a nontoxic but sufficient amount of the agent to provide a beneficial effect. The amount of active agent that is “effective” will vary from subject to subject, depending on the age and general condition of the individual, the particular active agent or agents, and the like. Unless otherwise indicated, the term “therapeutically effective” amount as used herein is intended to encompass an amount effective for the prevention of an adverse condition and/or the amelioration of an adverse condition, i.e., in addition to an amount effective for the treatment of an adverse condition.

As will be apparent to those of skill in the art upon reading this invention, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

Unless otherwise indicated, the invention is not limited to specific formulation components, modes of administration, anti-inflammatory agents, manufacturing processes, or the like, as such may vary.

Unless otherwise defined, all technical and scientific terms used herein have the same meaning as commonly understood by one of ordinary skill in the art to which this invention pertains. In the case of conflict, the present document, including definitions will control.

Definitions

Chronic Wound: A chronic wound may be defined as any wound that is failing to heal as anticipated or that has been stuck in any one phase of wound healing for a period of six weeks or more. (Collier, M. (2002) Wound bed preparation (NT Plus Wound Care Supplement) Nursing Times 98:2, 55-57.) Chronic wounds result from an alteration in one or more of the phases of normal wound healing and may be caused by cellular imbalances. Examples of such cellular imbalances are increased levels of enzymes or matrix metalloproteinases (MMPs), a decreased number of available active macrophages and/or a decreased number of “active” growth factors. (Calvin M (1998) Cutaneous wound repair. Wounds 10:12-32). It is now thought that most chronic wounds, such as chronic leg ulcers, are stuck in the phase known as early inflammation. (Falanga V. et al., Workshop on the pathogenesis of chronic wounds. J Invest Dermatol 1994; 102 (1): 125-127.)

Sometimes the inflammation process can go on too long. Since the inflammation phase can damage surrounding tissues, such an extended phase can result in a cycle of damage, inflammation, and more damage caused by inflammation. This greatly delays wound healing and can result in what is called a chronic wound.

Soft tissue damage: Soft tissue damage occurs through direct or indirect trauma to muscles, ligaments, and joint capsules. Usually, direct trauma refers to an injury occurring from blunt trauma or a sudden overload, and is known as macrotrauma, i.e., true muscle tear or ligament sprain. In contrast, indirect trauma results from repeated submaximal loading, leading to clinical signs and symptoms. Injury presents itself in three stages: acute, subacute/overuse, and acute/chronic. Soft tissue damages, like trying sport performances (marathon), sport injuries, contusions, large burns and frost-bites, post-operative inflammations, etc. Inflammation has been found to play a pathological role in soft tissue damage.

Arthritis: Arthritis is inflammation of one or more joints, which results in pain, swelling, stiffness, and limited movement. There are over 100 different types of arthritis. Arthritis involves the breakdown of cartilage usually resulting in inflammation. In chronic arthritis, the inflammation does not go away after healing of an injury and results in long-term pain and deformity. Osteoarthritis is the most common type and is more likely to occur with age. Psoriasis is a common, chronic skin condition that causes red patches on the body. About 1 in 20 people with psoriasis will develop arthritis with the skin condition. In most cases, psoriasis comes before the arthritis. Rheumatoid arthritis (RA) is an autoimmune disease that causes chronic inflammation of the joints. Rheumatoid arthritis can also cause inflammation of the tissue around the joints, as well as in other organs in the body.

Acute wounds: A wound occurs when the integrity of any tissue is compromised. A wound may be caused by an act, such as a gunshot, fall, or surgical procedure; by an infectious disease; or by an underlying condition. Acute wounds have a wide range of causes. Acute wounds include surgical incisions and traumatic injuries such as lacerations, abrasions, avulsions, penetrations or bites, and burn injuries. The general symptoms of a wound are localized pain and bleeding. Acute wounds normally proceed through an orderly and timely reparative process that results in sustained restoration of anatomic and functional integrity. If an acute wound fails to heal within six weeks, it can become a chronic wound.

IBD: Inflammatory bowel disease (IBD) is a group of inflammatory conditions of the colon and small intestine. The major types of IBD are Crohn's disease and ulcerative colitis. The main difference between Crohn's disease and UC is the location and nature of the inflammatory changes. Crohn's can affect any part of the gastrointestinal tract, from mouth to anus (skip lesions), although a majority of the cases start in the terminal ileum. Ulcerative colitis, in contrast, is restricted to the colon and the rectum. Microscopically, ulcerative colitis is restricted to the mucosa (epithelial lining of the gut), while Crohn's disease affects the whole bowel wall (“transmural lesions”). While IBD can limit quality of life because of pain, vomiting, diarrhea, and other socially unacceptable symptoms, it is rarely fatal on its own. IBD is treated by locally administering drugs with high anti-inflammatory effects, such as prednisone.

Anti-inflammatory agent: An anti-inflammatory agent is a drug or substance that reduces inflammation (redness, swelling, and pain) in the body. Anti-inflammatory agents block certain substances in the body that cause inflammation. They are used to treat many different conditions. Such agents are used for the treatment of inflammation and disorders, diseases, and adverse conditions, i.e., pathologies, caused by or otherwise associated with inflammatory processes.

Pain, heat, redness, and swelling (dolor, calor, rubor, tumor) are classic

manifestations of the inflammatory process. Abnormalities of the joints of the spine, associated muscles, tendons, ligaments and bone structural abnormalities can all result in pain and need for neurosurgical consultations. Typically, patients will not require immediate surgical intervention, and therefore require treatments to reduce pain and enhance quality of life activities. (Marienfeld R, et al. Eur J Immunol. 1997 July; 27 (7): 1601-9).

In most cases, the genesis of pain is inflammatory, regardless of the etiology. With the elucidation of the role of inflammatory cytokines, there is now a clear understanding of the pathways by which many anti-inflammatory drugs can alleviate inflammation and relieve pain. Prostaglandins are a family of chemicals that are produced by the cells of the body and have several important functions. They promote inflammation that is necessary for healing, but also results in pain, and fever. Prostaglandins are produced within the body's cells by the enzyme cyclooxygenase (COX).

The use of non-steroidal anti-inflammatory drug (NSAID) medication is still the mainstay of most classically taught clinicians for inflammatory pain. NSAIDs block the COX enzymes (COX-1 and COX-2) and reduce prostaglandins throughout the body. As a consequence, ongoing inflammation, pain, and fever are reduced. NSAID mechanisms are primarily through interaction with proinflammatory cytokines interleukin (IL)-1a, IL-1b, IL-6 and tumor necrosis factor (TNF-α). Increased concentrations of TNF-α are believed to cause the cardinal signs of inflammation to occur. Examples of FDA approved NSAIDs are aspirin, celecoxib (CELEBREX®), diclofenac (CAMBIAR, CATAFLAM®, VOLTAREN-XR®, ZIPSOR®, ZORVOLEX®), etodolac (LODINE®), ibuprofen (MOTRIN®, Advil®), indomethacin (INDOCIN®), ketoprofen, naproxen (ALEVER, ANAPROX®, NAPRELAN®, NAPROSYN®), oxaprozin (DAYPRO®), piroxicam (FELDENE®), salsalate (DISALSATE®) and tolmetin (TOLECTIN®).

On the other hand, there are analgesics that are commonly associated with anti-inflammatory drugs but that have no anti-inflammatory effects. An example is paracetamol (known as acetaminophen or Tylenol in the U.S.). As opposed to NSAIDs, which reduce pain and inflammation by inhibiting COX enzymes, paracetamol has been shown to block the reuptake of endocannabinoids, which only reduces pain, likely explaining why it has minimal effect on inflammation; paracetamol is sometimes combined with an NSAID (in place of an opioid) in clinical practice to enhance the pain relief of the NSAID while still receiving the injury/disease modulating effect of NSAID-induced inflammation reduction (which is not received from opioid/paracetamol combinations).

Use of anti-inflammatory and/or pain-reducing agents have several side-effects. Long-term use of NSAIDs can cause gastric erosions, which can become stomach ulcers and in extreme cases can cause severe hemorrhage, resulting in death. The risk of death as a result of GI bleeding caused by the use of NSAIDs is 1 in 12,000 for adults aged 16-45. The risk increases almost twentyfold for those over 75. Other dangers of NSAIDs are exacerbating asthma and causing kidney damage. Apart from aspirin, prescription and over-the-counter NSAIDs also increase the risk of heart attack and stroke

Unfortunately, most anti-inflammatory and/or pain-reducing agents are insoluble or have poor solubility in water, which slows down the transport of these agents to sites within the body, and thereby delays and reduces relief afforded by such agents. Examples are dexamethasone, ibuprofen, diclofenac sodium and methyl salicylate.

Further, most, if not all anti-inflammatory and/or pain-reducing agents have severe side-effects. Long-term use of NSAIDs can cause gastric erosions, which can become stomach ulcers and in extreme cases can cause severe hemorrhage, resulting in death. Other dangers of NSAIDs are exacerbating asthma and causing kidney damage. Apart from aspirin, prescription and over-the-counter NSAIDs also increase the risk of heart attack and stroke For example, the risk of death from gastro-intestinal bleed is as high as 17%.

| | | | | |
|---|---|---|---|---|
| Age | Number | Number | Chance of GI | Chance of dying |
| range | taking | with GI | bleed due to | from GI bleed due |
| (years) | NSAID | bleed | NSAID | to NSAID |

Risk in any one year is 1 in:

| | | | | |
|---|---|---|---|---|
| 16-45 | 2100 | 1 | 2100 | 12353 |
| 45-64 | 3230 | 5 | 646 | 3800 |
| 65-74 | 2280 | 4 | 570 | 3353 |
| >75 | 1540 | 14 | 110 | 647 |

Data from AL Blower, et al. Emergency admissions for upper gastrointestinal disease and their relation to NSAID use. Aliment Pharmacol. Ther. 1997 11:283-91 adjusted for 100,000 people.

The enhanced solubility of the agents in the presence of MSM as disclosed herein, allows use of reduced amounts of drugs and thus dramatically reduces harmful side-effects associated with these agents.

Chelating agent: Chelation is a chemical combination with a metal in complexes in which the metal is part of a ring. An organic ligand is called a chelator or chelating agent, the chelate is a metal complex. The larger number of ring closures to a metal atom the more stable is the compound. The stability of a chelate is also related to the number of atoms in the chelate ring. Monodentate ligands which have one coordinating atom like H₂O or NH₃ are easily broken apart by other chemical processes, whereas polydentate chelators, donating multiple binds to metal ion, provide more stable complexes. Chlorophyll, a green plant pigment, is a chelate that consists of a central magnesium atom joined with four complex anti-inflammatory agent (pyrrole ring). Heme is an iron chelate which contains iron (II) ion in the center of the porphyrin. Chelating agents offer a wide range of sequestrants to control metal ions in aqueous systems. By forming stable water soluble complexes with multivalent metal ions, chelating agents prevent undesired interaction by blocking normal reactivity of metal ions. EDTA (ethylenediamine tetraacetate) is a good example of common anti-inflammatory agents which have nitrogen atoms and short chain carboxylic groups.

Examples of chelators of iron and calcium include, but are not limited to, diethylene triamine pentaacetic acid (DTPA), ethylene diamine tetraacetic acid (EDTA), nitrilotriacetic acid (NTA), 1,3-propylene diamine tetraacetic acid (PDTA), Ethylene diamine disuccinic acid (EDDS), and ethylene glycol tetraacetic acid (EGTA). Any suitable chelating agent known in the art, which is biologically safe and able to chelate iron, calcium or other metals, is suitable for the invention.

Compounds useful as chelating agents herein include any compounds that coordinate to or form complexes with a divalent or polyvalent metal cation, thus serving as a sequestrant of such cations. Accordingly, the term “chelating agent” herein includes not only divalent and polyvalent ligands (which are typically referred to as “chelators”) but also monovalent ligands capable of coordinating to or forming complexes with the metal cation. Preferred chelating agents herein, however, are basic addition salts of a polyacid, e.g., a polycarboxylic acid, a polysulfonic acid, or a polyphosphonic acid, with polycarboxylates particularly preferred. The anti-inflammatory agent generally represents about 0.6 wt. % to 10 wt. %, preferably about 1.0 wt. % to 5.0 wt. %, of the formulation.

Suitable biocompatible chelating agents useful in conjunction with the present invention include, without limitation, monomeric polyacids such as EDTA, cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, pharmaceutically acceptable salts thereof, and combinations of any of the foregoing. Other exemplary chelating agents include: phosphates, e.g., pyrophosphates, tripolyphosphates, and hexametaphosphates.

EDTA and ophthalmologically acceptable EDTA salts are particularly preferred, wherein representative ophthalmologically acceptable EDTA salts are typically selected from diammonium EDTA, disodium EDTA, dipotassium EDTA, triammonium EDTA, trisodium EDTA, tripotassium EDTA, and calcium disodium EDTA.

In some embodiments, the chelating agent incorporated in the formulation is a prochelator. A prochelator is any molecule that is converted to a chelator when exposed to the appropriate chemical or physical conditions. For example, BSIH (isonicotinic acid [2-(4,4,5,5-tetramethyl-[1,3,2]dioxaborolan-2-yl)-benzylidene]-hydrazide) prochelators are converted by hydrogen peroxide into SIH (salicylaldehyde isonicotinoyl hydrazone) iron-chelating agents that inhibit iron-catalyzed hydroxyl radical generation.

Immediately after injury during the process of establishing hemostasis, the cells surrounding the injury release calcium, which is utilized in the clotting of blood. (Steinckwich N, et al., Potent inhibition of store-operated Ca2+ influx and superoxide production in HL60 cells and polymorphonuclear neutrophils by the pyrazole derivative BTP2. J Leukoc Biol 2007, 81:1054-1064; Moses S, et al., Smooth muscle cell response to mechanical injury involves intracellular calcium release and ERK1/ERK2 phosphorylation. Exp Cell Res. 2001; 269:88-96). After this process is over, early inflammation is characterized by the presence and active participation of neutrophils (in which Calcium plays a key role) in the attempt by the body to control pathogens. (Hauser C J, et al., Major trauma enhances store-operated calcium influx in human neutrophils. J Trauma. 2000 April; 48 (4): 592-7; discussion 597-8.) The neutrophils utilize the extracellular calcium to generate superoxide ions via the NADPH oxidase pathway, and the superoxide attacks both the “pathogens” as well as the surrounding healthy tissue. (Brechard S., Tschirhart E. J. Regulation of superoxide production in neutrophils: Role of calcium influx. J. Leuk. Biol. 2008). The compromised healthy tissue leads to a further influx of neutrophils, and the wound expands. At some point, the extracellular calcium is depleted, resulting in a reduction of both superoxide production, and the reduction of cell death by apoptosis or necrosis, and the inflammation subsides. Once the inflammation subsides, the regenerative/proliferative stage can take over, and the wound begins to heal.

Most chronic wounds occur in diabetics, or in patients suffering from a compromised circulatory system. Typically, chronic wounds are preceded by edemataceous limbs, characterized by a pitting edema. (Wolf M, Cutaneous and Subcutaneous Lesions Of The Lower Limbs In Connection With The Veins, Differential Diagnosis. Archives of Dermatology and Syphilology, 1945). The edema is the result of increased fluid efflux from the circulatory system, and a reduction in lymph flow. This fluid in the extracellular limb is calcium and iron rich, and produces Reactive Oxygen Species (ROS) under conditions such as, after the occurrence of an injury.

Thermal injuries trigger an intense local and systemic inflammatory response. While it is transient systemically, burn wounds suffer from the effects of acute influx of inflammatory mediators and growth factors for an extended time. Burn therapies focus on reducing this inflammatory response, intending to limit the progression of burns.

It is generally accepted that inflammation is the body's coordinated form of defense against the internal and external influences, and it has a key role in maintaining the homeostasis of the cells and organism. In case of immunogenic inflammation, monocyte or macrophage or dendritic cells have an important role in detection of foreign substances and in the production of pro-inflammatory cytokines responsible for creating inflammation. In case of neurogenic inflammation, the sensory nerves sense the foreign influences leading to the production of pro-inflammatory neuropeptides.

However, in most cases the inflammatory reaction can be so strong that, independent of the original cause, the inflammation by itself causes damage to the body. Inflammation results in increased metabolism, which leads to the release of an extreme quantity of cell-and tissue-damaging reactive oxygen species (ROS). This explains why the reduction of inflammation goes together with the improvement of the diseased condition, and with the decrease of the damages. There is a role of inflammation not merely in infections, allergies and (auto) immune diseases, but also in degenerative diseases characterized by cell loss (apoptosis) and in tumors characterized by cell proliferation where inflammation precedes or causes the degenerative disease or the tumor.

The present invention provides methods and formulations for the treatment of chronic wounds, acute wounds or burn conditions by enhancing the solubility of an anti-inflammatory agent, to ensure that an enhanced amount of the agent effective to reduce inflammation reaches the intended target within the body.

The formulations of the present invention facilitate the solubility in water of active anti-inflammatory agents. In acute injuries, burn damage, arthritis and other such conditions the formulations are suitable for topical administration. Where the inflammation is in an internal organ, such as in the colon or the intestines, the formulation can also be delivered locally-typically by ingestion or injection of tablets, gels, solutions, suspensions and the like.

Solubility enhancer: The solubility enhancer is selected to enhance the water-solubility of an anti-inflammatory agent for enhanced delivery to the tissues, extra-cellular matrices, and/or cell membranes of a body. An “effective amount” of the solubility enhancer represents an amount and concentration within a formulation of the invention that is sufficient to provide a measurable increase in the penetration of an anti-inflammatory agent through one or more of the localized sites of chronic wound, acute wound or burn injury in a subject than would otherwise be the case without the inclusion of the solubility enhancer within the formulation.

In certain instances, the solubility enhancer may be present in a formulation of the invention in an amount that ranges from about 0.01 wt. % or less to about 30 wt. % or more, typically in the range of about 0.1 wt. % to about 20 wt. %, more typically in the range of about 1 wt. % to about 11 wt. %, and most typically in the range of about 2 wt. % to about 8 wt. %, for instance, 5 wt. %.

The solubility enhancer is generally of the formula (I)

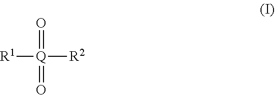

wherein R¹ and R² are independently selected from C₂-C₆ alkyl, C₁-C₆ heteroalkyl, C₆-C₁₄ aralkyl, and C₂-C₁₂ heteroaralkyl, any of which may be substituted, and Q is S or P. Compounds wherein Q is S and R¹ and R² are C₁-C₃ alkyl are preferred, with methylsulfonylmethane (MSM) being the optimal solubility enhancer.

The phrase “having the formula” or “having the structure” is not intended to be limiting and is used in the same way that the term “comprising” is commonly used. With respect to the above structure, the term “alkyl” refers to a linear, branched, or cyclic saturated hydrocarbon group containing 1 to 6 carbon atoms, such as methyl, ethyl, n-propyl, isopropyl, n-butyl, isobutyl, t-butyl, cyclopentyl, cyclohexyl and the like. If not otherwise indicated, the term “alkyl” includes unsubstituted and substituted alkyl, wherein the substituents may be, for example, halo, hydroxyl, sulfhydryl, alkoxy, acyl, etc. The term “alkoxy” intends an alkyl group bound through a single, terminal ether linkage; that is, an “alkoxy” group may be represented as —O-alkyl where alkyl is as defined above. The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 14 carbon atoms. Exemplary aryl groups are contain one aromatic ring or two fused or linked aromatic rings, e.g., phenyl, naphthyl, biphenyl, diphenylether, diphenylamine, benzophenone, and the like. “Aryl” includes unsubstituted and substituted aryl, wherein the substituents may be as set forth above with respect to optionally substituted “alkyl” groups. The term “aralkyl” refers to an alkyl group with an aryl substituent, wherein “aryl” and “alkyl” are as defined above. Preferred aralkyl groups contain 6 to 14 carbon atoms, and particularly preferred aralkyl groups contain 6 to 8 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The term “acyl” refers to substituents having the formula —(CO)-alkyl, —(CO)-aryl, or —(CO)-aralkyl, wherein “alkyl,” “aryl”, and “aralkyl” are as defined above. The terms “heteroalkyl” and “heteroaralkyl” are used to refer to heteroatom-containing alkyl and aralkyl groups, respectively, i.e., alkyl and aralkyl groups in which one or more carbon atoms is replaced with an atom other than carbon, e.g., nitrogen, oxygen, sulfur, phosphorus or silicon, typically nitrogen, oxygen or sulfur.

Suitable solubility enhancers include methylsulfonylmethane (MSM; also referred to as methyl sulfone) and/or combinations of MSM with dimethylsulfoxide (DMSO). MSM is an odorless, highly water-soluble (34% w/v at 79° F.) white crystalline compound with a melting point of 108-110° C. and a molecular weight of 94.1 g/mol. MSM is thought to serve as a multifunctional agent herein, insofar as the agent not only increases the permeability of biological membranes such as cell membranes, but may also facilitate the transport of one or more formulation components throughout the layers of the skin (i.e., epidermis, dermis and subcutaneous fat layers), as well as across mucus membranes, endothelial layers, and the like. Furthermore, it has been suggested that MSM per se has medicative effects, and can serve as an anti-inflammatory agent as well as an analgesic. It has been also suggested that MSM also acts to improve oxidative metabolism in biological tissues, and is a source of organic sulfur, which may assist in the reduction of scarring. MSM additionally possesses beneficial solubilization properties, in that it is soluble in water, as noted above, but exhibits both hydrophilic and hydrophobic properties because of the presence of polar S═O groups and nonpolar methyl groups. The molecular structure of MSM also allows for hydrogen bonding with other molecules, i.e., between the oxygen atom of each S═O group and hydrogen atoms of other molecules, and for formation of van der Waals associations, i.e., between the methyl groups and nonpolar (e.g., hydrocarbyl) segments of other molecules. The methods and formulations herein may involve use of two or more solubility enhancers used in combination.

Suitable solubility enhancers include, by way of example, methylsulfonylmethane (MSM; also referred to as methyl sulfone), combinations of MSM with dimethylsulfoxide (DMSO), or a combination of MSM and, in a less preferred embodiment, DMSO, with MSM particularly preferred. DMSO, a solubility enhancer but essentially a solvent, is not particularly suitable for formulations according to this invention. DMSO works as a highly potent solvent and therefore a carrier of its solutes. In contrast, MSM works in a totally different manner by forming hydrogen bonds with select molecules and changing their charge characteristics of the target molecule allowing the target molecule to get through charged barriers like biologic membranes.

There are differences in chemical structures between MSM and DMSO. Methylsulfonylmethane (MSM) is an organosulfur compound with the formula (CH₃)₂SO₂. It is also known by several other names including DMSO₂, methyl sulfone, and dimethyl sulfone. This colorless solid features the sulfonyl functional group and is considered relatively inert chemically. MSM has the structure:

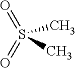

Dimethyl sulfoxide (DMSO) on the other hand is an organosulfur compound with the formula (CH₃)₂SO. This colorless liquid is a widely-used polar aprotic solvent that dissolves both polar and nonpolar compounds and is miscible in a wide range of organic solvents as well as water. DMSO has the structure:

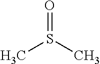

MSM is an odorless, highly water-soluble (34% w/v@79° F.) white crystalline compound with a melting point of 108-110° C. and a molecular weight of 94.1 g/mol. MSM serves as a multifunctional agent herein, insofar as the agent not only increases cell membrane permeability, but also acts as a “transport facilitating agent” (TFA) that aids in the transport of one or more formulation components to oral tissues. Furthermore, MSM per se provides medicating effects, and can serve as an anti-inflammatory agent as well as an analgesic. MSM also acts to improve oxidative metabolism in biological tissues, and is a source of organic sulfur, which assists in the reduction of scarring. MSM additionally possesses unique and beneficial solubilizing properties, in that it is soluble in water, as noted above, but exhibits both hydrophilic and hydrophobic properties because of the presence of polar S=O groups and nonpolar methyl groups. The molecular structure of MSM also allows for hydrogen bonding with other molecules, i.e., between the oxygen atom of each S=O group and hydrogen atoms of other molecules, and for formation of van der Waal associations, i.e., between the methyl groups and nonpolar (e.g., hydrocarbon) segments of other molecules. Ideally, the concentration of MSM in the present formulations is in the range of about 0.1 wt. % to 40 wt. %, or from about 1 wt. % to about 4, 5, 6, 7, 8, 10, 15 wt. %, and preferably between about 1.5 wt. % to 8.0 wt. %.

Topical NSAIDs (Non-steroidal anti-inflammatory drugs) which contain non-steroidal anti-inflammatory drugs, vanilloid receptor antagonists, neuronal calcium channel blockers and other anti-inflammatoires known in the art, such as diclofenac (Voltaren®, Cataflam®), cis-capsaicin (Civamide®), ibuprofen, naproxen, etc in topical form, are incorporated in certain embodiments of the formulation.

In addition to such anti-inflammatory agents, a formulation of the invention may also include any botanical species that has anti-inflammatory properties, such as hyssop, ginger, etc. Arnica montana (which contains helenalin), a sesquiterpene lactone, turmeric (which contains curcumin), cannabis or cannabinoid (such as CBD) and willow bark, which contains salicylic acid, a substance related to the active ingredient in aspirin. These herbs are encompassed by the present invention and one or more herbs can be combined in a formulation with one or more solubility enhancers.

The concentrations of the solubility enhancer and anti-inflammatory agent in the formulation are also of interest. In general, enhancer concentrations on the order of a few percent by weight may be used in liquid formulations for topical administration, for example in the range of about 0.01 wt. % or less to about 30 wt. % or more, typically in the range of about 0.1 wt. % to about 15 wt. %, more typically in the range of about 1 wt. % to about 11 wt. %, and most typically in the range of about 2 wt. % to about 8 wt. %, for instance, 5 wt. %. In liquid formulations for topical administration, the concentration of anti-inflammatory agent will also, generally, be within a therapeutically acceptable ranges. A representative such formulation contains about 5 wt. % MSM and in the range of about 0.01 wt. % to about 10 wt. % anti-inflammatory agent. In one particular embodiment, the formulation comprises the anti-inflammatory or pain-relieving agent, 5.4% MSM and 2.6% EDTA.

Without being bound by theory, it is believed that the solubility enhancer in formulations of the invention assists in the process of transporting enhanced amounts of the anti-inflammatory agent not just into the tissue, but across biological membranes and to the site at which the active agent is needed.

The subject invention provides methods of using a formulation, which includes a solubility enhancer and a anti-inflammatory agent, to topically apply the formulation at a localized site of a burn, chronic wound or other such inflammatory conditions, such as to translocate the anti-inflammatory agent across a biological membrane of a cell.

Accordingly, in one embodiment, the invention provides a method of transporting a anti-inflammatory agent (which may also be a test agent) into a cell. The agent is to be contacted with a cell. The cell may be provided in vitro, in vivo, ex vivo, or the like. Accordingly, the cell may be one that has been removed from a tissue and/or from the body, may be present within a tissue (such as within an organ) of the body (which organ or tissue may be present within the body or removed therefrom), or the cell may be one that has been removed from the body with the expectation that the cell is to be returned to the body, for instance, a gamete cell. The method further includes contacting the cell with a solubility enhancer.

Hence, a formulation of the present invention uses a solubility enhancer, such as methyl sulfonyl methane (MSM), for increasing the water solubility of biologically-active agents such as anti-inflammatory agents, across cellular membranes, for enhanced delivery, e.g., intracellular delivery, of beneficial agent to a cell. Hence, the invention also provides a method of introducing increased amounts of an anti-inflammatory agent of interest into a cell or a cell nucleus. The method includes contacting the cell with a formulation including a solubility enhancer and an anti-inflammatory agent, in an amount sufficient to enable efficient penetration into the cells. As stated above, in general, the method may be used for in vivo or in vitro internalization of the anti-inflammatory agent. For example, the formulation including the anti-inflammatory agent may be provided in vitro, ex vivo, or in vivo.

Accordingly, in certain instances, a method for transporting a biologically active or test agent into a cell is provided. The method may include contacting a cell with a solubility enhancer, e.g., methyl sulfonyl methane, and an anti-inflammatory agent under conditions sufficient to effect transport of the agent into the cell.

A variety of means can be used to formulate the compositions of the invention. Techniques for formulation and administration may be found in “Remington: The Science and Practice of Pharmacy,” Twentieth Edition, Lippincott Williams & Wilkins, Philadelphia, PA (1995). For human or animal administration, preparations should meet sterility, pyrogenicity, general safety and purity standards comparable to those required by the FDA. Administration of the pharmaceutical formulation can be performed in a variety of ways, as described herein.

The anti-inflammatory agent may be administered, if desired, in the form of a salt, ester, crystalline form, hydrate, or the like, provided it is pharmaceutically acceptable. Salts, esters, etc. may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992).

The amount of anti-inflammatory agent administered will depend on a number of factors and will vary from subject to subject and depend on the particular anti-inflammatory agent, the particular disorder or condition being treated, the severity of the symptoms, the subject's age, weight and general condition, and the judgment of the prescribing physician. The term “dosage form” denotes any form of a pharmaceutical composition that contains an amount of anti-inflammatory agent and solubility enhancer sufficient to achieve a therapeutic effect with a single administration or multiple administrations. The frequency of administration that will provide the most effective results in an efficient manner without overdosing will vary with the characteristics of the particular active agent, including both its pharmacological characteristics and its physical characteristics, such as hydrophilicity. The formulations of the present invention can also be formulated for controlled release or sustained release. The term “controlled release” refers to a pharmaceutical formulation in which release of the anti-inflammatory agent is not immediate, e.g., with a “controlled release” formulation, administration does not result in immediate release of the drug into an absorption pool. The term is used interchangeably with “nonimmediate release” as defined in Remington: The Science and Practice of Pharmacy, cited previously. In general, the term “controlled release” as used herein includes sustained release and delayed release formulations.

The term “sustained release” (synonymous with “extended release”) is used in its conventional sense to refer to a pharmaceutical formulation that provides for gradual release of an active agent over an extended period of time, and which preferably, although not necessarily, results in substantially constant blood levels of a drug over an extended time period.

The present formulations may also include conventional additives such as opacifiers, antioxidants, fragrance, colorant, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the methyl and propyl esters of p-hydroxybenzoic acid (i.e., methyl and propyl paraben), sodium benzoate, sorbic acid, imidurea, and combinations thereof.

Depending on the intended mode of administration, the pharmaceutical formulation may be a solid, semi-solid or liquid, such as, for example, a liquid, a cream, a suspension, an emulsion, beads, a powder, or the like, preferably in unit dosage form suitable for single administration of a precise dosage. Suitable pharmaceutical formulations and dosage forms may be prepared using conventional methods known to those in the field of pharmaceutical formulation and described in the pertinent texts and literature, e.g., in Remington: The Science and Practice of Pharmacy, cited previously herein.

The dosage regimen will depend on a number of factors that may readily be determined, such as severity of the condition and responsiveness of the condition to be treated, but will normally be one or more doses per day, with a course of treatment lasting from a single dose to multiple doses over a day or several days to several months, or until a cure is effected or a diminution of disease state or other adverse condition is achieved.

The formulations are preferably administered topically to a subject in need of treatment. The term “topical administration” is used in its conventional sense to mean delivery (e.g., process of applying or spreading one or more compositions according to the instant disclosure onto the surface of the skin) to a predetermined area of skin or mucosa of a subject, as in, for example, the treatment of various skin disorders. Topical administration, in contrast to transdermal administration, is intended to provide a local rather than a systemic effect. In certain instances, as may be stated or implied by the circumstances, the terms “topical drug administration” and “transdermal drug administration” may be used interchangeably.

Typically, the formulations are suitable for being administered in topical lotion, gel, cream, ointment, injection, implant, dermal patch, or medicated bandage form, suitable for application on and around the localized burn or wound site.

Gels formulated with these anti-inflammatoires were tested at peak solubility levels against existing topical formulations for anti-inflammatory properties and pain relief. It was found that symptomatic relief happened in 1/10th to ½ the time, and this was despite the concentrations of the said anti-inflammatory agents being much lower than the existing topical formulations.

The following examples are put forth so as to provide those skilled in the art with a complete invention and description of how to make and use embodiments in accordance with the invention, and are not intended to limit the scope of what the inventors regard as their discovery. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. All publications and patent applications cited in this specification are herein incorporated by reference as if each individual publication or patent application were specifically and individually indicated to be incorporated by reference.

The increases in solubility in water of certain representative anti-inflammatory agents in formulations comprising MSM (about 5.4% w/w; range between 0.1 wt. % to about 15 wt. %, typically around 5%) are as follows:

Dexamethsone solubilty increases from insoluble to ˜250 mg per ml of water.

Ibuprofen solubility increases from insoluble to ˜150 mg per ml of water

Diclofenac increases from 0.004 mg to 50 mg per ml of water.

Methyl salicylate solubility increases from 0.00065 mg to 0.013 mg per ml of water

Gels formulated with these anti-inflammatoires were tested at peak solubility levels against existing topical formulations for anti-inflammatory properties and pain relief. It was found that symptomatic relief happened in 1/10th to ½ the time.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claim.

What is claimed is:

1. A aqueous pharmaceutical formulation comprising:
(a) an anti-inflammatory and/or pain-reducing agent that is insoluble or has low solubility in water,
(b) a solubility enhancer, wherein the solubility enhancer is provided in an amount sufficient to increase solubility of the anti-inflammatory agent by at least 20% sufficient to provide symptomatic relief of inflammation and/or pain in a subject when the formulation is administered locally and
(c) optionally, a pharmaceutically acceptable excipient.

2. The pharmaceutical formulation of claim 1, wherein the solubility enhancer is methylsulfonylmethane (MSM).

3. The pharmaceutical formulation of claim 1, wherein the inflammation and/or pain is associated with a condition selected from a burn, an acute wound, a chronic wound, a radiation-induced injury, a soft tissue injury, irritable bowel disease, osteoarthritis, and rheumatoid arthritis.

4. The pharmaceutical formulation of claim 2, wherein the symptomatic relief is manifested in 1/10th to ½ the time required in the absence of the solubility enhancer, MSM.

5. The pharmaceutical formulation of claim 2, wherein the amounts of MSM is between 0.01% and 10% by weight of the formulation.

6. The pharmaceutical formulation of claim 2, wherein the anti-inflammatory and/or pain reducing drug is effective at dosage amounts lower than the standard prescribed amount in the absence of MSM.

7. The pharmaceutical formulation of claim 2, wherein the formulation displays reduced side-effects associated with the anti-inflammatory and/or pain reducing drug as compared to side-effects associated with the standard prescribed amount of the drug in the absence of MSM.

8. The pharmaceutical formulation of claim 2, wherein the solubility of the anti-inflammatory and/or pain reducing drug in water is increased between 10 and 20,000 fold.

9. The pharmaceutical formulation of claim 2, wherein the anti-inflammatory and/or pain reducing drug is insoluble in water.

10. The pharmaceutical formulation of claim 2, wherein the anti-inflammatory and/or pain reducing drug is an NSAID selected from aspirin, celecoxib (CELEBREX®), diclofenac (CAMBIA®, CATAFLAM®, VOLTAREN-XR®, ZIPSOR®, ZORVOLEX®), etodolac (LODINE®), ibuprofen (MOTRIN®, Advil®), indomethacin (INDOCIN®), ketoprofen, naproxen (ALEVE®, ANAPROX®, NAPRELAN®, NAPROSYN®), oxaprozin (DAYPRO®), piroxicam (FELDENE®), salsalate (DISALSATE®) and tolmetin (TOLECTIN®).

11. The pharmaceutical formulation of claim 1, further comprising a chelator or a prochelator, or a combination thereof.

12. The pharmaceutical formulation of claim 11, wherein the chelator is selected from the group consisting of diethylene triamine pentaacetic acid (DTPA), ethylene diamine tetraacetic acid (EDTA), cyclohexanediamine tetraacetic acid (CDTA), hydroxyethylethylenediamine triacetic acid (HEDTA), nitrilotriacetic acid (NTA), 1,3-propylene diamine tetraacetic acid (PDTA), ethylene diamine disuccinic acid (EDDS), ethylene glycol tetraacetic acid (EGTA), dimercaptopropane sulfonic acid (DMPS), dimercaptosuccinic acid (DMSA), aminotrimethylene phosphonic acid (ATPA), citric acid, pharmaceutically acceptable salts thereof, and combinations thereof.

13. The pharmaceutical formulation of claim 1, wherein the anti-inflammatory and/or pain-reducing agent is from a natural source.

14. The pharmaceutical formulation of claim 1, wherein the formulation is a topical lotion, gel, ointment, injection, implant, dermal patch, or medicated bandage, suitable for application on an injured site.

15. The pharmaceutical formulation of claim 1, wherein the formulation is a tablet, caplet, gel, suspension, solution, suitable for localized delivery to a site affected by inflammation by ingestion or injection.

16. The pharmaceutical formulation of claim 1, wherein the anti-inflammatory and/or pain-reducing agent is an anaesthetic selected from aminoamides and aminoesters.

17. The pharmaceutical formulation of claim 16, wherein the aminoamide is selected from articaine, bupivacaine, cinchocaine, etidocaine, levobupivacaine, lidocaine, mepivacaine, prilocalne, ropivacaine, and trimecaine.

18. The pharmaceutical formulation of claim 16, wherein the aminoester is selected from benzocaine, chloroprocaine, cyclomethycaine, dimethocaine, piperocaine, propoxycaine, procaine, proparacaine, and tetracaine
